(19) United States
(12) Patent Application Publication
(10) Pub. No.: US 2026/0133158 A1
(43) Pub. Date: May 14, 2026

(54) SYSTEM AND METHOD FOR DETERMINING A CONCENTRATION OF AN ANALYTE IN A CARRIER GAS OF A QUASI-BINARY OR BINARY GAS MIXTURE

(71) Applicant: Drager Safety AG & Co. KGaA, Lubeck (DE)

(72) Inventors: Peter Tschuncky, Lübeck (DE); Marcus Oertel, Lübeck (DE); Rolf Eckhardt, Lübeck (DE); Tobias Reier, Lübeck (DE); Henrik Veelken, Lübeck (DE)

(21) Appl. No.: 19/386,366

(22) Filed: Nov. 12, 2025

(30) Foreign Application Priority Data
Nov. 14, 2024 (DE) 102024133368.5

(51) Int. Cl. G01N 27/416; G01N 27/413; G01N 31/00; G01N 33/00

(52) U.S. Cl. CPC G01N 27/4163; G01N 27/413; G01N 31/005; G01N 33/0006

(57) ABSTRACT

A system and a method for determining a concentration of an analyte in a carrier gas of a quasi-binary or binary gas mixture are provided. The system comprise an electrochemical gas sensor. The electrochemical gas sensor comprises a working electrode and a counter electrode. The electrochemical gas sensor is configured by means of the working electrode to oxidize and/or reduce the analyte and to reduce and/or oxidize at least one component of the carrier gas, and to provide a corresponding measurement signal. The system further comprises a memory unit in which calibration information for the gas mixture is stored, and a data processing unit which is configured to: receive the measurement signal, receive the calibration information, determine the concentration of the analyte in the carrier gas from the measurement signal, and, from the calibration information, provide the determined concentration of the analyte in the carrier gas.

### CROSS-REFERENCE TO RELATED APPLICATIONS

This application claims the priority of German Patent Application No. 102024133368.5, filed on Nov. 14, 2024, and titled “SYSTEM AND METHOD FOR DETERMINING A CONCENTRATION OF AN ANALYTE IN A CARRIER GAS OF A QUASI-BINARY OR BINARY GAS MIXTURE”, which is hereby incorporated by reference in its entirety for all non-limiting purposes.

### TECHNICAL FIELD

The present disclosure relates to a system and to a method for determining a concentration of an analyte in a carrier gas of a quasi-binary or binary gas mixture.

### BACKGROUND

Systems and methods for determining the concentration of an analyte in a carrier gas of a quasi-binary or binary gas mixture are generally known.

Given the increasing importance of alternatives to fossil fuels, hydrogen production is gaining in importance. In this field, electrolyzers are used to produce oxygen (O2) and hydrogen (H2) from water (H2O). In electrolyzers, the proper functioning of the electrolysis cells must be monitored at the electrodes. For this purpose, the cathode gas, which mainly contains hydrogen, is monitored to ensure that little or no oxygen is present in order to avoid malfunction of the electrolyzer or the formation of an explosive oxyhydrogen mixture, which can occur particularly at higher pressures on the anode side than on the cathode side. Naturally, high H2 concentrations (90 to 100 vol. %) occur. An explosive gas mixture occurs at approximately 95% H2 and 5% O2. The gas mixture in the stationary state is a binary gas mixture. Its composition must be monitored without introducing an ignition source into the process.

Accordingly, there is a need to be able to determine the concentration of the oxygen in the produced hydrogen and/or the concentration of the hydrogen in the produced oxygen in a simple manner. In both cases, the mixture is a binary gas mixture. In this context, it may also be necessary to determine the concentration of oxygen or hydrogen in another carrier gas, which may be a gas mixture such as air. In this example, therefore, the gas mixture is a quasi-binary gas mixture.

DE 20 2009 003 553 U1 describes a way to identify gases and to experimentally determine gas quantities and the compositions of binary gas mixtures. This also applies to low-boiling liquids and binary liquid mixtures at a pressure below the saturation vapor pressure. For this purpose, an acoustic resonance tube is filled with the corresponding gas, the binary gas mixture, or with a low-boiling liquid or a binary liquid mixture, and standing waves are generated with a loudspeaker in the range between 0 and several kHz. The resonance frequency at which the standing waves develop is a function of the type of gas, the type of gas mixture, or the low-boiling liquid or the binary liquid mixture, the temperature, the length of the resonance tube and the total pressure.

Furthermore, the use of sensors that investigate the thermal conductivity of the gas mixture is known for the analysis of binary or quasi-binary gas mixtures. An example of such a sensor is the CALOMAT 7 sensor from Siemens. The measuring principle of this device is the measurement of the thermal conductivity of the binary gas mixture. The thermal conductivity of H2 is 0.1805 W·m-1·K-1 and of O2 is 0.02658 W·m-1·K-1. With the resulting thermal conductivity of the gas mixture, the mixing ratio of O2 and H2 can be determined, for example, approximately linearly or with the mixing formula of Mason and Saxena. In this case, both thermal conductivities are positive and the thermal conductivity of the mixture lies between the thermal conductivities of the individual components.

### SUMMARY

The present disclosure is based on the object of providing an alternative system and corresponding method for determining a concentration of an analyte in a carrier gas of a quasi-binary or binary gas mixture, in particular a system and corresponding method with improved sensitivity.

This object is achieved by the system and by the method described herein.

The claims, the figures and the description provide further advantageous embodiments of the present disclosure.

According to the present disclosure, a system is provided for determining a concentration of an analyte in a carrier gas of a quasi-binary or binary gas mixture. The system comprises an electrochemical gas sensor, wherein the electrochemical gas sensor has a working electrode and a counter electrode, wherein the electrochemical gas sensor is configured by means of the working electrode to oxidize and/or reduce the analyte and to reduce and/or oxidize at least one component of the carrier gas, and to provide a corresponding measurement signal. The system further comprises a memory unit in which calibration information for the gas mixture is stored, and a data processing unit which is configured to: receive the measurement signal, receive the calibration information, determine the concentration of the analyte in the carrier gas from the measurement signal, and, from the calibration information, provide the determined concentration of the analyte in the carrier gas.

In this way, it is possible to determine a concentration of the analyte in the carrier gas of the quasi-binary or binary gas mixture by means of an electrochemical gas sensor which reacts to both the analyte and, at least partially, to the carrier gas at the working electrode—and only using newly generated calibration information for a quasi-binary or binary gas mixture. This enables, in particular, the use of known electrochemical gas sensors and thus reduces the effort required to deploy the system. Furthermore, the simultaneous reaction of analyte and at least part of the carrier gas can lead to an amplification of the measurement signal, which can improve the sensitivity of the system. This occurs when the analyte reacts reductively or oxidatively and at least one component of the carrier gas reacts oxidatively or reductively complementarily at the working electrode.

A gas mixture is understood to be a mixture of gases, namely a mixture of the analyte being tested with the carrier gas. A binary gas mixture is a mixture of the analyte with a pure gas as carrier gas, such as a mixture of O2 as analyte and H2 as carrier gas or a mixture of H2 as analyte and O2 as carrier gas. A quasi-binary gas mixture is a mixture of the analyte with a gas mixture as carrier gas, such as a mixture of O2 as analyte and air as carrier gas or a mixture of H2 as analyte and air as carrier gas, in which at least one component of the carrier gas can react at the working electrode and the remaining components of the carrier gas are electrochemically inert at the working electrode (at the same working potential). For a quasi-binary gas mixture, according to the present disclosure the composition of the carrier gas must be known, for example in the form of predetermined information.

The electrochemical gas sensor can be configured in a known manner and can optionally comprise, in addition to the working electrode and counter electrode, further electrodes, such as a further working electrode and/or a further counter electrode and/or a reference electrode.

The measurement signal can, for example, be a measuring current or a measuring voltage or a signal correlating with a measuring current or a measuring voltage.

The calibration information can be configured, for example, as a look-up table, as a function to be analytically evaluated or as other information that indicates a calibration curve.

The determination of the concentration of the analyte in the carrier gas from the measurement signal and from the calibration information can be carried out, for example, by (possibly direct) comparison of the measurement signal with the calibration information. Further signal processing and/or data processing steps can optionally be performed, e.g., to condition the measurement signal or to obtain intermediately processed values from the measurement signal that are suitable for comparison with the calibration information.

The data processing unit can, for example, be configured as a component of a gas measuring device, which may include the electrochemical gas sensor or can be designed to accommodate the electrochemical gas sensor.

In another example, the data processing unit may be designed as a handheld device, such as a smartphone or a computer, and may be configured to receive the measurement signal in a wired or wireless manner.

The memory unit can be configured, for example, as a component of a gas measuring device, a handheld device such as a smartphone, or as a component of a computer.

The measurement signal can be provided via a data interface for further data processing and/or as an output via a human-machine interface such as a display.

Providing the measurement signal may comprise determining an alarm condition depending on the determined concentration of the analyte in relation to a critical concentration of the analyte and, upon determining that the alarm condition exists, issuing an alarm via an alarm device such as a siren.

In some examples, the calibration information indicates a calibration curve in a coordinate system, wherein in the coordinate system the concentration of the analyte or a variable corresponding to the concentration of the analyte forms an abscissa and the measurement signal or a variable corresponding to the measurement signal forms an ordinate, wherein the calibration curve does not pass through a coordinate origin of the coordinate system.

This represents a particularly simple computational way of providing the calibration information.

For example, the offset of the calibration curve from the coordinate origin can be at least ±50 μA to ±800 μA. Whether the offset is configured to be positive or negative depends on whether the analyte is oxidized or reduced at the working electrode.

In some examples, the calibration curve is configured as a calibration line with a positive ordinate axis intercept and a negative slope.

Alternatively, the calibration curve is in some examples configured as a calibration line with a negative ordinate axis intercept and with a positive slope.

In some examples, the analyte is oxygen or hydrogen, wherein the carrier gas is—complementary thereto—hydrogen or oxygen, or wherein the carrier gas comprises—complementary thereto—hydrogen or oxygen.

In some examples, the electrochemical gas sensor further comprises a reference electrode and a protective electrode.

This can prevent gas that does not react at the working electrode from entering the gas sensor and shifting a reference potential of the reference electrode.

According to the present disclosure, a method for determining a concentration of an analyte in a carrier gas of a quasi-binary or binary gas mixture is further provided. The method comprises the steps of: receiving a measurement signal, wherein the measurement signal is obtainable by means of oxidation and/or reduction of the analyte and reduction and/or oxidation of at least one component of the carrier gas at a common working electrode, receiving calibration information for the gas mixture, determining the concentration of the analyte in the carrier gas from the measurement signal and from the calibration information, and providing the determined concentration of the analyte in the carrier gas.

The method has advantageous effects comparable to those of the system. All features disclosed in connection with the system are also deemed to be disclosed in connection with the method, and vice versa.

The method is in particular a computer-implemented method.

In some examples, the common working electrode is the working electrode of the described gas sensor and the data processing unit of the system is configured to carry out some or all steps of the method.

These and further advantages and features of the present disclosure can be found in the following description of the drawings.

### BRIEF DESCRIPTION OF THE DRAWINGS

FIG. 1 shows an embodiment of a system according to one or more example arrangements,

FIG. 2 shows embodiments of calibration curves,

FIG. 3 shows a profile of a measurement signal over time by way of example, which is obtainable with the system according to the one or more example arrangements of FIG. 1, and

FIG. 4 shows an embodiment of a method according to one or more example arrangements which can be carried out by means of the system according to FIG. 1.

### DETAILED DESCRIPTION

According to the present disclosure, a system 1000 is provided for determining a concentration c of an analyte in a carrier gas of a quasi-binary or binary gas mixture G.

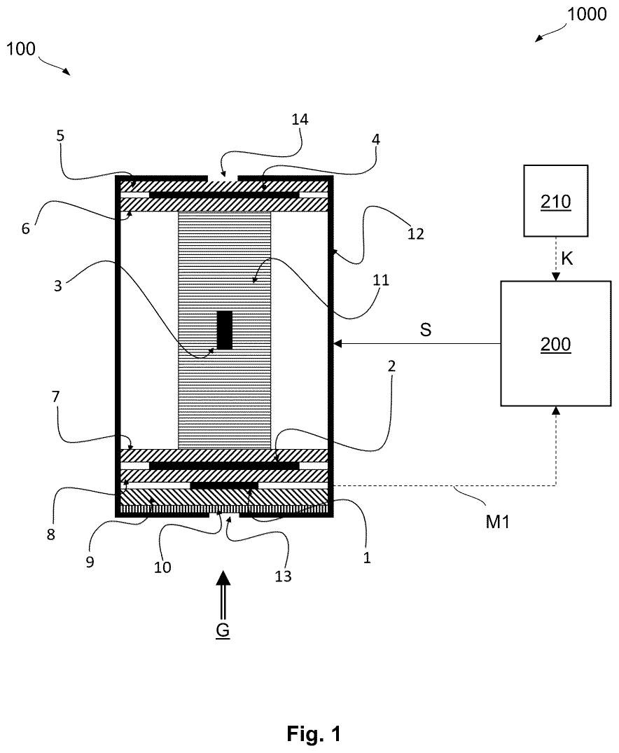

An embodiment of a system 1000 according to the present disclosure is shown in FIG. 1. The system 1000 comprises an electrochemical gas sensor 100, wherein the electrochemical gas sensor 100 has at least one working electrode 1 and one counter electrode 4. The electrochemical gas sensor 100 is configured by means of the working electrode 1 to oxidize and/or reduce the analyte and to reduce and/or oxidize at least one component of the carrier gas (complementarily) and to provide a corresponding measurement signal M1. The system 1000 further comprises a memory unit 210 in which calibration information K for the gas mixture G is stored. The system 1000 also comprises a data processing unit 200 which is configured to receive the measurement signal M1, to receive the calibration information K, to determine the concentration c of the analyte in the carrier gas from the measurement signal M1 and from the calibration information K, and to provide the determined concentration C of the analyte in the carrier gas.

The data processing unit 200 can be configured to control an operation of the electrochemical gas sensor 100 by means of a signal S.

The electrochemical gas sensor 100 may include additional components.

As shown in FIG. 1, the electrochemical gas sensor 100 may include a housing 12 that houses some or all of the components of the electrochemical gas sensor 100.

The gas mixture G can enter the electrochemical gas sensor 100 through a housing opening 13 and a membrane 10. The material of the membrane 10 is, in some examples, a polymer of perfluoroalkoxy (PFA), polytetrafluoroethylene (PTFE), perfluoroethylenepropylene (FEP), polyvinylidene fluoride (PVDF), polyethylene (PE) or polypropylene (PP). The thickness of the material can be between 1 μm and 100 μm. The membrane 10 is, in some examples, liquid-tight and gas-permeable and can function as a material flow limiting membrane.

Viewed in the gas inlet direction (see arrow of the gas mixture G in FIG. 1), a further supporting membrane 9 can optionally be arranged behind the membrane 10. It can serve to mechanically support the membrane 10, since this is typically very thin. The material of the supporting membrane 9 comprises, for example, porous PTFE, PE or PP. The membrane 9 can also be configured as a carrier of the working electrode 1. It is also possible to make the membrane 10 thicker and to dispense with the use of the supporting membrane 9.

The working electrode 1 can be configured as a platinum metal-containing electrode and preferably as a sintered electrode. The sensor reaction takes place at the working electrode 1. For example, if the analyte is oxygen and the carrier gas is hydrogen, at the working electrode 1, depending on the electrode potential, oxygen is reduced and hydrogen is oxidized, for example in accordance with the following electrode equation:

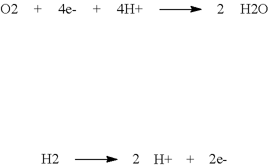

The measurement signal M1 obtained from the measuring current can be used to determine, for example, the oxygen content in the hydrogen.

According to the present disclosure, both the analyte and the carrier gas or a component of the carrier gas electrochemically react at the working electrode 1 at the same time. The current flow (measuring current) of the working electrode 1 and thus the measurement signal M1 is then the sum of the current flows of the analyte and the carrier gas and/or the component of the carrier gas. The contributions of the individual currents from the analyte reaction and the carrier gas reaction may have the same or different signs. The weighting of the contributions is represented by the corresponding calibration information K. The calibration information K takes into account the reaction of both gases (analyte and carrier gas) and is used to convert the current flow or the measurement signal M1 into the concentration c of the analyte. In a binary or quasi-binary gas mixture, both contents—analyte fraction and carrier gas fraction—can be determined.

The electrochemical gas sensor 100 according to the present disclosure has the advantage during operation that the presence of the analyte in the binary or quasi-binary gas mixture G displaces a corresponding portion of carrier gas and thereby reduces the signal of the carrier gas. On the other hand, depending on the analyte and carrier gas, the reductive or oxidative component of the measuring current corresponding to the analyte component, which results from the analyte reaction at the working electrode 1, increases with increasing analyte portion. Due to these mutually reinforcing effects, the electrochemical gas sensor 100 reacts very sensitively to analytes present. Compared to the displacement of one gas by another gas, this opposite reaction leads to a relative amplification of the signal if the analyte is oxygen or hydrogen and the carrier gas is—complementary thereto—hydrogen or oxygen, or the carrier gas comprises complementary hydrogen or oxygen. This is compared to a gas mixture of, for example, hydrogen and nitrogen, in which the nitrogen would not react but would only reduce the hydrogen signal by displacing it.

The system 1000 according to the present disclosure thus has a significantly simpler structure than conventional systems based on a different sensor technology. Furthermore, the resulting measurement signal obtainable with the system 1000 according to the present disclosure has an amplification due to the opposing reaction mechanisms for a measurement of O2 in H2 or H2 in O2, so that the sensitivity of the system 1000 is improved.

Viewed in the gas inlet direction, a further membrane 8 can optionally be arranged behind the working electrode 1. The membrane 8 can fulfill two functions. On the one hand, the membrane 8 can ensure wetting of the working electrode 1 and the optional protective electrode 2, which will be described later, with electrolytes in order to connect them together in an electrochemical half-cell system. On the other hand, the membrane 8 prevents electrical short circuits between the working electrode 1 and the optional protective electrode 2. The membrane 8 can be provided, for example, as a nonwoven made of mineral and/or organic fibers. Nonwovens made of glass fibers or polyolefin have proven to be particularly suitable.

Optionally, the electrochemical gas sensor 100 may have a protective electrode 2. The protective electrode 2 may in some examples be provided from the same material as the working electrode 1. Furthermore, the protective electrode 2 can in some examples be operated in the same potential range, most preferably at an identical potential as the working electrode 1. It is shown that, in some examples, the protective electrode 2 has a larger face than the working electrode 1. The protective electrode 2 serves to react gas that does not react at the working electrode 1, e.g., H2 or O2, and thus to prevent these gases from penetrating into the rear space of the sensor and shifting the reference potential of the optional reference electrode 3. Compared to a 3-electrode sensor, this allows for permanently stable measuring behavior. In particular, when hydrogen is present as part of the binary gas mixture, there is the problem that hydrogen is more permeable than other gases and can therefore easily penetrate into the electrochemical gas sensor 100 without being completely consumed by the working electrode 1. This may result in an enrichment of hydrogen inside the electrochemical gas sensor 100 and lead to an unstable measurement signal.

Viewed in the gas inlet direction behind the optional protective electrode 2, the electrochemical gas sensor 100 may further comprise further membranes 6 and 7. These membranes 6 and 7 can be configured with the same function and, if necessary, material as the membrane 8.

A further membrane 11 can be arranged between the membranes 6 and 7, which can act as a support core element and thus cause mechanical contact between the components of the electrochemical gas sensor. The further membrane 11 can also act as a wick for the electrolyte. The support core 11 can be configured, for example, as a glass sintered body or as a plastic sleeve with an electrolyte-absorbing core.

The electrochemical gas sensor 100 may further optionally comprise a reference electrode 3.

The counter electrode 4 can be provided, for example, as a sintered electrode made of a noble metal, preferably platinum. At the counter electrode 4, a reaction takes place that is opposite to that of the working electrode 1, thereby ensuring the electroneutrality of the cell as a whole (the electrochemical gas sensor 100). Possible reactions at the counter electrode 4 are:

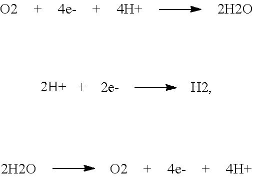

The electrochemical gas sensor 100 may further optionally have an additional membrane 5 acting as a pressure equalization valve. The further membrane 5 can be arranged in or on a housing opening 14 of the electrochemical gas sensor 100. The further membrane 5 can be provided from porous PTFE. The further membrane 5 can, on the one hand, carry the counter electrode 4 and, on the other hand, ensure that gases formed at the counter electrode 4 can leave the electrochemical gas sensor 100. In addition, the further membrane 5 can provide pressure equalization through the housing opening 14 of the electrochemical gas sensor 100.

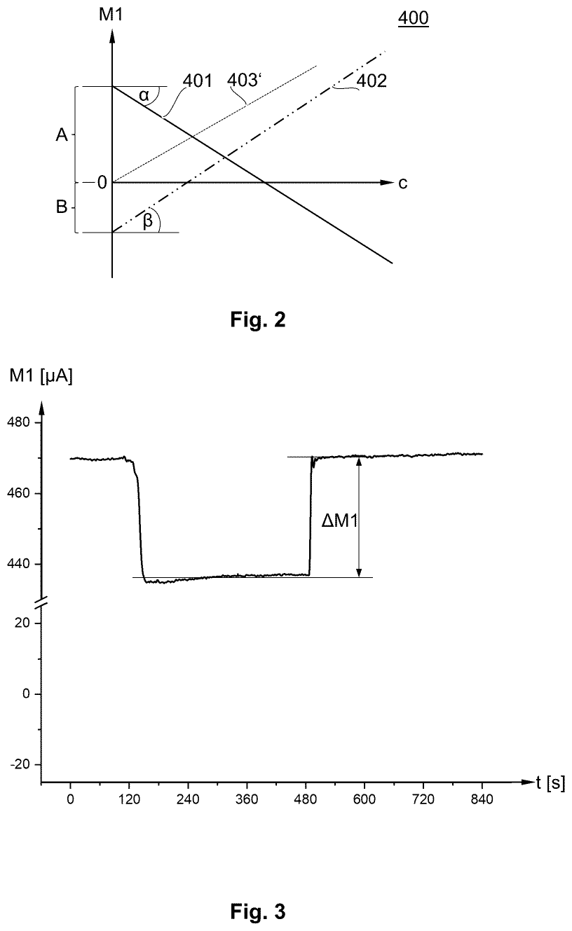

In FIG. 2, three calibration lines 401, 402, 403′ are shown as embodiment examples of calibration information K in a coordinate system 400. The calibration lines 401, 402 are in accordance with the present disclosure, while the calibration line 403′ represents a calibration line of a conventional electrochemical gas sensor from the prior art that is not in accordance with the present disclosure.

It can be seen from FIG. 2 that the calibration information K can thus indicate a calibration curve 401, 402, e.g., a calibration line 401, 402, in a coordinate system 400, wherein in the coordinate system 400 the concentration of the analyte c or a variable corresponding to the concentration of the analyte c forms an abscissa and the measurement signal M1 or a variable corresponding to the measurement signal M1 forms an ordinate, wherein the calibration curve 401, 402 does not pass through a coordinate origin 0 of the coordinate system 400.

Depending on whether the analyte reacts oxidatively or reductively at the working electrode 1, the calibration curve 401 can be designed as a calibration line 401 with a positive ordinate axis intercept A and with a negative slope α, or the calibration curve 402 can be configured as a calibration line 402 with a negative ordinate axis intercept B and with a positive slope β.

The parameters A, α, B and β are obtainable by means of a calibration and can be stored as part of the calibration information K in the memory unit 210.

The calibration can be performed, for example, by means of the following procedure. The electrochemical gas sensor 100 can be biased with the working potential and gassed with 100 vol. % carrier gas (e.g., H2). Once a saturation value is reached, this value is calibrated with 0 vol. % analyte (e.g., O2). See FIG. 2. It is clear that when c=0, M1>>0 (if the analyte is, for example, O2) or M1<<0 (if the analyte is, for example, H2 and the carrier gas is, for example, O2). A defined binary or quasi-binary gas mixture G can then be created, for example, with 96 vol. % H2 as carrier gas and 4 vol. % O2 as analyte. Due to the displacement of the H2 and the resulting lower sensor current and the reduction of O2 at the working electrode 1 (negative current), the sensor current for 4 vol. % O2 is as follows:

$${I}_{4 Vol \% O 2} = {I}_{96 Vol \% H 2} + {I}_{4 Vol \% O 2} \\ {I}_{0} - {I}_{4 Vol \% O 2} = {I}_{100 Vol \% H 2} - {I}_{96 Vol \% H 2} - \vert {I}_{4 Vol \% O 2} \vert \\ where {I}_{100 Vol \% H 2} > {I}_{96 Vol \% H 2} >> 0 ∧ {I}_{4 Vol \% O 2} < 0$$

FIG. 3 shows a profile according to the present disclosure of a measurement signal M1 by way of example over time t, which was obtained using a system 1000 according to the present disclosure. In the example shown, the system 1000 was first gassed with 100 vol. % carrier gas, namely H2. The resulting measurement signal M1 is approximately 470 μA. At approximately time t=120 s, a gas mixture G consisting of carrier gas and analyte was supplied to the system 1000. This resulted in a signal drop ΔM1 of approximately 35 μA. Using the calibration information K, it was determined that the changed measurement signal M1 of approximately 435 μA corresponds to a concentration of the analyte in the carrier gas of approximately 4%.

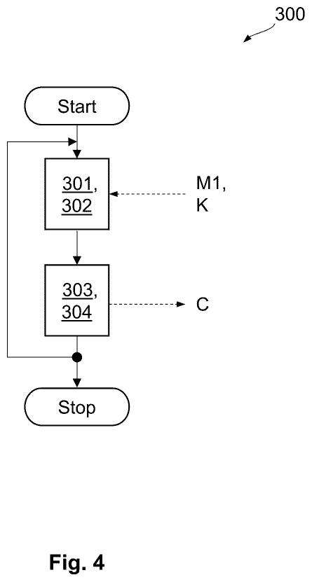

FIG. 4 shows an embodiment of a method 300 according to the present disclosure for determining a concentration c of an analyte in a carrier gas of a quasi-binary or binary gas mixture G.

The method 300 comprises the step 301: receiving a measurement signal M1, wherein the measurement signal M1 is obtainable by means of oxidation and/or reduction of the analyte and reduction and/or oxidation of at least one component of the carrier gas of a common working electrode 1.

The method 300 comprises step 302: receiving calibration information K for the gas mixture G.

The method 300 comprises step 303: determining the concentration c of the analyte in the carrier gas from the measurement signal M1 and from the calibration information K.

The method 300 includes step 304: providing the determined concentration C of the analyte in the carrier gas.

All features disclosed herein can be combined with one another as desired as long as this is not contradictory or relates to alternatives.

### LIST OF REFERENCE SIGNS

1 Working electrode
2 Protective electrode
3 Reference electrode
4 Counter electrode
5 Membrane
6 Membrane
7 Membrane, non-woven separator
8 Membrane, non-woven separator
9 Membrane, supporting membrane
10 Membrane, diffusion limiter
11 Membrane, support core
12 Housing
13 Gas inlet opening
14 Pressure equalization opening
100 Gas sensor
200 Data processing unit
210 Memory unit
300 Method
301, 302, . . . Steps of the method
400 Coordinate system
401, 402 Calibration curve
403′ Calibration curve in accordance with the prior art
1000 System
A First axis intercept
α First slope
B Second axis intercept
β Second slope
c Concentration of the analyte
C Specific concentration of the analyte
G Gas, gas mixture
K Calibration information
M1 Measurement signal
S Signal

1-7. (canceled)

8. A system for determining a concentration of an analyte in a carrier gas of a gas mixture, comprising:
an electrochemical gas sensor,
wherein the electrochemical gas sensor comprises a working electrode and a counter electrode,
wherein the electrochemical gas sensor is configured by means of the working electrode to:
perform at least one of:
oxidizing the analyte, or
reducing the analyte;
perform at least one of:
reducing at least one component of the carrier gas, or
oxidizing the at least one component of the carrier gas; and
provide a measurement signal;
a memory unit storing calibration information for the gas mixture; and
a data processing unit configured to:
receive the measurement signal;
receive the calibration information;
determine the concentration of the analyte in the carrier gas based on the measurement signal and based on the calibration information; and
provide the determined concentration of the analyte in the carrier gas.

9. The system of claim 8, wherein the gas mixture is:
a binary gas mixture, or
a quasi-binary gas mixture.

10. The system of claim 8, wherein:
the calibration information indicates a calibration curve in a coordinate system,
in the coordinate system:
the concentration of the analyte or a variable corresponding to the concentration of the analyte forms an abscissa; and
the measurement signal or a variable corresponding to the measurement signal forms an ordinate, and
the calibration curve does not pass through a coordinate origin of the coordinate system.

11. The system of claim 10, wherein:
the calibration curve is configured as a calibration line with a positive ordinate axis intercept and with a negative slope, or
the calibration curve is configured as a calibration line with a negative ordinate axis intercept and with a positive slope.

12. The system of claim 8, wherein:
the analyte is oxygen and the carrier gas is hydrogen, or
the analyte is hydrogen and the carrier gas is oxygen.

13. The system of claim 8, wherein:
the analyte is hydrogen and the carrier gas comprises oxygen, or
the analyte is oxygen and the carrier gas comprises hydrogen.

14. The system of claim 8, wherein the electrochemical gas sensor further comprises a protective electrode and a reference electrode.

15. The system of claim 8, wherein the system is configured to provide the determined concentration of the analyte in the carrier gas via a display interface.

16. The system of claim 8, wherein the electrochemical gas sensor further comprises an additional membrane that acts as a pressure equalization valve.

17. The system of claim 8, wherein the electrochemical gas sensor is further configured to receive, via a liquid impermeable membrane, the gas mixture.

18. A method for determining a concentration of an analyte in a carrier gas of a gas mixture, comprising:
receiving, by a data processing unit, a measurement signal,
wherein the measurement signal corresponds to one or more of:
oxidation of the analyte, or
reduction of the analyte, and
wherein the measurement signal further corresponds to one or more of:
reduction of at least one component of the carrier gas, or
oxidation of the at least one component of the carrier gas,
at a common working electrode;
receiving calibration information for the gas mixture;
determining the concentration of the analyte in the carrier gas based on the measurement signal and based on the calibration information; and
providing the determined concentration of the analyte in the carrier gas.

19. The method of claim 18, wherein the gas mixture is:
a binary gas mixture, or
a quasi-binary gas mixture.

20. The method of claim 18, wherein:
the calibration information indicates a calibration curve in a coordinate system,
in the coordinate system:
the concentration of the analyte or a variable corresponding to the concentration of the analyte forms an abscissa; and
the measurement signal or a variable corresponding to the measurement signal forms an ordinate, and
the calibration curve does not pass through a coordinate origin of the coordinate system.

21. The method of claim 20, wherein:
the calibration curve is configured as a calibration line with a positive ordinate axis intercept and with a negative slope, or
the calibration curve is configured as a calibration line with a negative ordinate axis intercept and with a positive slope.

22. The method of claim 18, wherein:
the analyte is oxygen and the carrier gas is hydrogen, or
the analyte is hydrogen and the carrier gas is oxygen.

23. The method of claim 18, wherein:
the analyte is oxygen and the carrier gas comprises hydrogen, or
the analyte is hydrogen and the carrier gas comprises oxygen.

24. The method of claim 18,
wherein the common working electrode is a working electrode of an electrochemical gas sensor, wherein the electrochemical gas sensor further comprises a counter electrode and wherein the method further comprises:
performing, by the electrochemical gas sensor, at least one of:
oxidizing the analyte, or
reducing the analyte;
performing at least one of:
reducing the at least one component of the carrier gas, or
oxidizing the at least one component of the carrier gas; and
providing the measurement signal.

25. The method of claim 24, wherein the electrochemical gas sensor further comprises a protective electrode and a reference electrode.

26. The method of claim 18, wherein the providing the determined concentration of the analyte in the carrier gas comprises outputting the determined concentration of the analyte via a display interface.

27. The method of claim 18, further comprising receiving, by an electrochemical sensor, before the receiving the measurement signal, and via a liquid impermeable membrane, the gas mixture.
